(19) United States
(12) Patent Application Publication
(10) Pub. No.: US 2005/0267552 A1
(43) Pub. Date: Dec. 1, 2005

(54) Electrosurgical device

(75) Inventors: Laura Rozsi Conquergood, Etobicoke (CA); Krishan Shah, Mississauga (CA); Taylor Lloyd William Hillier, Toronto (CA)

Correspondence Address: OGILVY RENAULT LLP, 1981 MCGILL COLLEGE AVENUE, SUITE 1600, MONTREAL, QC, H3A2Y3, CA

(73) Assignee: Baylis Medical Company Inc., Montreal (CA)

(21) Appl. No.: 10/853,126

(22) Filed: May 26, 2004

(51) Int. Cl. A61F 7/00

(52) U.S. Cl. 607/96

(57) ABSTRACT

An electrosurgical device having improved placement accuracy is provided. The electrosurgical device comprises an elongate member having a proximal end, a distal end and a lumen therethrough, a conductive tip at the distal end for delivery of energy to the tissue, an electrical connector at or near the proximal end for flexibly coupling a power source control unit to supply energy to the conductive tip, a fluid delivery connection interface flexibly coupled at or near the proximal end for coupling a fluid delivery mechanism. The device further comprises a temperature sensor. A method of delivering electrical energy to a target on an animal is also provided.

The present invention relates to electrosurgical devices and more particularly to devices used to deliver high or radio frequency electrical current to a target area in a body.

### BACKGROUND OF THE INVENTION

Electrosurgical procedures typically rely on the application of high frequency or radio frequency (“RF”) electrical power to treat, cut, ablate or coagulate tissue structures. Such tissue structures may include neural tissue. The efficacy of the minimally invasive technique of delivering high frequency electrical current to neural tissue has been studied. Studies show that Radio Frequency (“RF”) lumbar facet denervation is an effective method of relieving low back pain. The high frequency electrical current is typically delivered from a generator through a probe that is placed in a patient's body via an introducer needle. The introducer needle includes an insulated shaft with an exposed electrically conductive tip at the distal end. A hub at the proximal end can also be provided as a connection site for an injection syringe. Introducer needles can also therefore be used to inject anesthetic fluid or other therapeutic agents. Tissue resistance to the high frequency electrical current at the conductive tip causes heating of adjacent tissue. The temperature is increased to a sufficient level to coagulate unmyelinated nerve structures, at which point a lesion is formed. This results in relief from pain.

Introducer needles with varying geometries are used in such applications. For example, the conductive tip of the introducer needle can be pointed, blunt and rounded or open, varying in shape in accordance with the needs of different procedures. Pointed tips are self-penetrating while rounded tips are useful in soft tissue areas such as the brain where it is critical not to damage nerves. However, blunt introducer needles can do more tissue damage than small diameter sharp introducer needles. U.S. Pat. No. 6,146,380 to Racz et al. describes introducer needles with curved conductive tips used in high frequency lesioning.

The probe is generally a stainless steel electrode that is manufactured to fit in the introducer needle. The probe is used to deliver the high frequency energy from the generator to the conductive tip of the introducer needle. Some probes incorporate a temperature sensor to allow for monitoring of the temperature throughout the procedure. The temperature can be used to control the delivery of high frequency energy. It is also known to utilize insulation on the probe itself thus making the conductive tip a component of the probe and not the introducer needle.

A known treatment procedure utilizes the introducer needle, having a hollow shaft and a removable stylet therein. This introducer needle is inserted into the patient's body and positioned via imaging technology. Once the introducer needle is positioned, the stylet is withdrawn. The distal end of the probe is inserted into the shaft of the introducer needle until the distal end of the probe is at least flush with the distal end of the shaft. The probe is connected to a generator that generates electrical current. To ensure that a lesion will be formed on the appropriate nerves, a stimulation procedure is employed. This involves delivery of low frequency electrical current that excites nerves. This procedure can differentiate between motor and sensory nerves and confirm that the nerve to be lesioned is in fact the source of pain.

After placement is confirmed with the stimulation procedure, the probe is withdrawn. Then a syringe is attached to the proximal end hub of the introducer needle to inject anesthetic fluid or other therapeutic agents. After which, the syringe is unattached and the probe is reinserted into the shaft of the introducer needle. Finally, high frequency electrical current is applied from the generator, through the probe and introducer needle to the tissue adjacent to the conductive tip and a lesion is formed. This high frequency electrical current returns to the generator through a return electrode typically placed on an exterior surface of the patient's body.

Such a procedure can be used to denervate very specific portions of a patient's spine. This procedure is also applied to other areas of anatomy such as intercostal and trigeminal nerves. Accurate placement of the introducer needle's conductive tip in a complicated structure like the spine requires great technical skill by the treating physician. In these procedures, the introducer needle is often viewed via X-ray or fluoroscopy to assist placement as it is guided into the body.

One limitation of this technique is that the placement achieved at the beginning of the procedure can be altered by the attachment of a fluid delivery mechanism, actuation of the fluid delivery mechanism or the replacement of the probe after the stimulation procedure is completed. For example, to ensure that the fluid being injected does not leak, the fluid delivery mechanism must be tightly secured to the hub of the introducer needle. This twisting or pushing motion applies pressure to the introducer needle, thus altering its placement within the body. Also, the probes are generally designed in such a manner that they are only slightly smaller than the inner diameter of the introducer needle to allow for a good electrical connection between the probe and the conductive tip region of the introducer needle. This tight fit requires relatively high insertion forces to align the distal end of the probe with the end of the introducer needle. Therefore, when the probe is inserted, removed or reinserted after the injection of therapeutic agent, the forces applied can move the introducer needle. Movement caused by any of these inherent procedural difficulties creates potential for unpredictable lesion sites. The range of distance the tip of the introducer needle may move depends on the depth of the needle and the flexibility of the tissue. The tip may move radially up to 5 mm and axially up to 10 mm. Even a couple of millimetres may cause the procedure to be ineffective or unsafe. Therefore, placement often relies on the physician to visually monitor the position of the conductive tip throughout the procedure. However, even slight variations in position can affect the outcome of the procedure. These variations can be so slight that the imaging technology available and physician may miss them. Repeating the stimulation procedure to confirm the position is not viable since anesthetic has already been introduced. Physicians can therefore only rely on their visual monitoring skills and attempt to be careful not to move the introducer needle.

Also, most traditional devices are constructed in such a manner for reuse following sterilization. However, these devices are often made with small components in which bio materials such as tissue and blood become lodged.

U.S. Pat. No. 6,464,661 to Edwards et al. describes a medical probe for treatment of tissue via insertion of a catheter through natural body cavities. It includes a temperature sensor and a conductive tip for delivery of radio frequency energy. This device is specifically designed for the treatment of prostate tissue and as such is a flexible catheter that is inserted through natural body cavities utilizing the RF energy to help with insertion. While a fluid supply lumen in the probe is contemplated, attachment of a syringe is not specifically disclosed. Properties of the Edwards invention indicate that it would be impossible to manufacture the device, described in U.S. Pat. No. 6,464,661, with the dimensions and features of this invention.

TOP Corporation 19-10 Senju Nakai-cho, Adachi-ku Tokyo 120-0035, Japan, manufactures Pole Needle—XE (23 G, 60 mm, active tip: 5 mm). This Pole Needle is for RF facet joint procedure and is a rigid hollow shaft ending in a conductive tip at the distal end. A flexible tubing is attached at the proximal end of the Pole needle. Wiring for delivery of the RF energy runs from an alligator clip down the flexible tubing to the conductive shaft. A treatment tube for delivery of a treatment fluid runs from a syringe connector, along the flexible tube and ends at the proximal end of the shaft. A lumen of the treatment tube is in fluid communication with a lumen of the shaft. The treatment fluid exits through an opening at the end of beveled conductive tip. The Pole Needle does not have the capacity to monitor temperature.

A need generally exists for an improved electrosurgical device.

### SUMMARY OF THE INVENTION

The present invention provides an electrosurgical device with improved positioning characteristics. According to one broad aspect of the invention, an electrosurgical device is provided for treating animal tissue. The device comprises an elongate member having a proximal end, a distal end and a lumen therethrough; a conductive tip at the distal end for delivering energy to the tissue; a power connector at the proximal end for flexibly coupling a power source to supply energy to the conductive tip; a temperature sensor at the distal end; and at least one fluid delivery interface connection flexibly coupled at the proximal end for coupling a fluid delivery mechanism to deliver a treatment composition through the lumen to the distal end.

In one embodiment of the invention, to facilitate precise placement of the exposed tip, the tip is distinguishable from the rest of the needle when viewed under X-rays and fluoroscopy by providing a cannula with a radiopaque marking.

According to another aspect of the invention, a method is provided for delivering energy to a predetermined treatment area of an animal body. The method comprises the steps of: (i) providing an electrosurgical device comprising: an elongate member having a proximal end, a distal end and a lumen therethrough; a conductive tip at the distal end for delivering energy to the tissue; a power connector at the proximal end for flexibly coupling a power source to supply energy to the conductive tip; a temperature sensor at the distal end; and at least one fluid delivery interface connection flexibly coupled at the proximal end for coupling a fluid delivery mechanism to deliver a treatment composition through the lumen to the distal end; ii) coupling the power source to the power connector; iii) coupling the fluid delivery mechanism to the fluid delivery interface connection; iv) positioning the device at or in the vicinity of the treatment area; v) administering the treatment composition; vi) delivering energy to the treatment area; vii) monitoring temperature at the treatment area; and viii) controlling the delivered energy based on the monitored temperature; whereby the steps iv)-vi) are performed without the necessity to remove a part of the electrosurgical device from the animal body.

### BRIEF DESCRIPTION OF THE DRAWINGS

These and other features of the embodiments of the invention will become more apparent in the following detailed description in which reference is made to the appended drawings wherein:

FIG. 1 is a side elevation view, fragmented, of an electrosurgical device in accordance with a first embodiment of the present invention, in a system.

FIG. 2 is an enlarged side elevation view of a conductive tip of an electrosurgical device in accordance with the first embodiment of the present invention.

FIG. 3 is a sectional side view of the handle of an electrosurgical device in accordance with the first embodiment of the present invention.

FIG. 4 is a sectional side view of the proximal end of the shaft of an electrosurgical device in accordance with the first embodiment of the invention where the wire exits the lumen of the shaft through an aperture.

FIG. 5A is a sectional front view of the shaft of an electrosurgical device in accordance with the first embodiment of the invention.

FIG. 5B is a sectional front view of the shaft of an electrosurgical device in accordance with a second embodiment of the invention, having a plurality of lumens in the shaft.

FIG. 6 is a side elevation view of a conductive tip of an electrosurgical device in accordance with a third embodiment of the invention having a curved tip, a radiopaque marker, and a textured surface on the shaft.

FIG. 7 is a side elevation view of an electrosurgical device in accordance with a fourth embodiment of the invention having a V-shaped handle.

FIG. 8A is a first partial flowchart of operations according to a preferred embodiment of a method aspect of the invention.

FIG. 8B is a second partial flowchart of operations according to the preferred embodiment of the method aspect of the invention.

### DESCRIPTION OF THE PREFERRED EMBODIMENTS

In the following description, numerous specific details are set forth to provide a thorough understanding of the invention. However, it is understood that the invention may be practiced without these specific details. In other instances, well-known structures and/or processes have not been described or shown in detail to not obscure the invention. In the description and drawings, like numerals refer to like structures or and/or processes.

The methods of the present invention are claimed and described herein as a series of steps. It should be understood that these methods and associated steps may be performed in any logical order. Moreover, the methods may be performed alone, or in conjunction with other procedures and treatments administered before, during or after such methods and steps set forth herein without departing from the scope and spirit of the invention. Further, it is contemplated that the term animals as used herein includes, but is not limited to, humans.

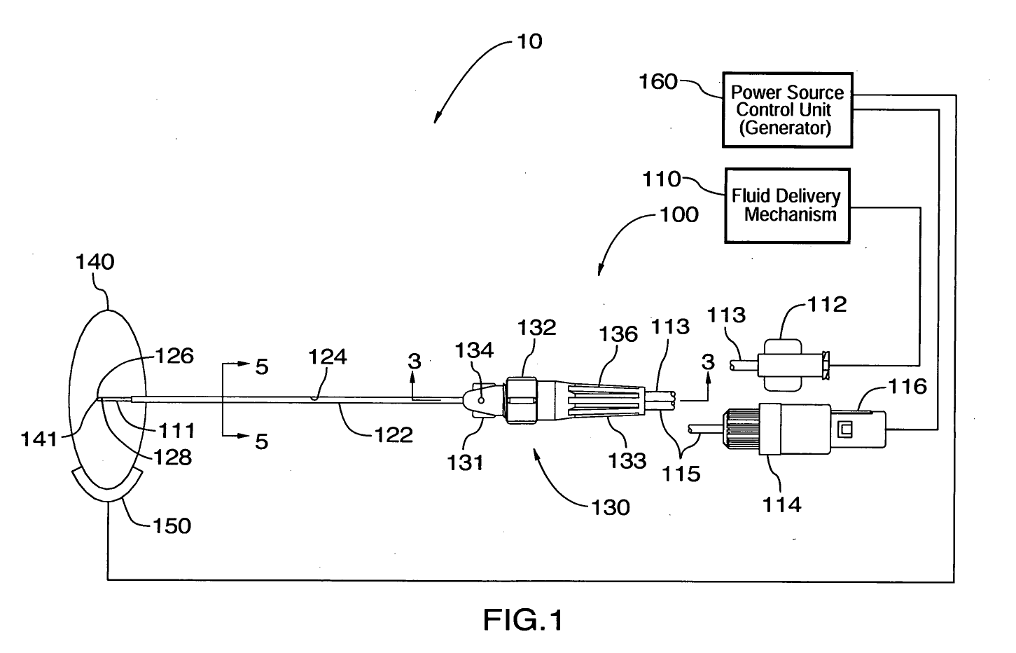

Referring to FIG. 1, a device 100 in accordance with one embodiment of the surgical device aspect of the invention, is shown in a system 10 for treating a body 140. System 10 comprises the electrosurgical device 100, a power source control unit 160, a return dispersive electrode 150, and a fluid delivery mechanism 110 for fluid composition injection such as, but not limited to, a syringe. Power source control unit 160 supplies energy to device 100, measures temperature feedback from a temperature sensor 126 of device 100, and provides impedance measurement between a conductive tip 111 of device 100 and the return dispersive electrode 150. Impedance measurement may be used during placement to target body tissue, which has specific electrical properties. Device 100 comprises a conductive shaft 124 and a handle 130. The conductive shaft 124 has an insulating coating 122 along a major portion of the outer length of the shaft 124 terminating adjacent the exposed conductive tip 111. Conductive tip 111 transmits energy to a target area 141 of body 140. In addition, conductive tip 111 may permit the instrument to penetrate into body 140 and navigate to desired target area 141. It will therefore be understood by a person skilled in the art that conductive tip 11 can be of varying dimensions and have various placements on the invention. For example, conductive tip 111 can be pointed, sharp and edged, blunt and rounded or open, varying in shape in accordance with the needs of different procedures. Also, while the preferred length of the conductive tip is between about 2 mm to about 10 mm, this length may vary depending on procedural requirements. Conductive tip 111 is preferably made of medical grade stainless steel, but other conductive surgical materials may be used.

Conductive shaft 124 of device 100 imparts rigidity to device 100 and facilitates the maneuvering of conductive tip 111 to reach the tissue to be ablated. In this embodiment of the invention, shaft 124 is hollow along its length defining a lumen. Shaft 124 may be used to transmit a therapeutic agent or treatment composition to conductive tip 111, as well as support any wiring for conductive tip 111. As well, an inner diameter of shaft 124 is sufficiently dimensioned to accommodate wiring for a temperature sensor 126 associated with the distal end of the shaft 124 about conductive tip 111. A preferred length of shaft 124 varies between about 5 cm to about 15 cm. It is understood however that the length may vary beyond this range according to the procedure.

Temperature sensor 126 for measuring the temperature at conductive tip 111 comprises a thermocouple, which includes wires running in the lumen of shaft 124 and insulated from the conductive shaft 124. Insulation may include either insulation on the inner wall of shaft 124 or insulation on the outer wall of the wires. The general use of a thermocouple to measure temperature is known in the art. The distal ends of the wires are minimally stripped of insulation. Conductive tip 111 with accompanying temperature sensor 126 may be formed by welding the distal ends of the wires of the thermocouple to shaft 124 at the distal end. Shaft 124 is then shaped such as by shaving or grinding the weld joint into a desired shape for the conductive tip 111. This shaving of the welded thermocouple joint reduces the thermal mass in the thermocouple junction and advantageously provides a faster response to temperature changes in the thermocouple.

Handle 130 has a flexible tube 113 coupled thereto in fluid communication with the lumen of shaft 124. A proximal end of flexible tube 113 is coupled to a fluid delivery interface connection 112. Handle 130 also provides a grip 136 for a user to manipulate device 100. Handle 130 is preferably medical grade injection moldable plastic or other materials that can be ethylene oxide sterilized. In other embodiments of the invention (not shown), handle 130 is not necessary and flexible tube 113 may be coupled directly to conductive shaft 124. Conductive tip 111 has an aperture 128, through which the treatment composition exits. Aperture 128 is formed in shaft 124 at a side thereof proximate conductive tip 111. The circumferential edge of aperture 128, on the outer wall of conductive tip 111, is rounded to prevent cutting of tissue while device 100 is inserted through body 140. Handle 130 has an aperture marker 134, in line with aperture 128 along the axis of shaft 124, to indicate the orientation of aperture 128 about the axis of shaft 124. Aperture marker 134 allows the user to target tissue for the delivery of treatment fluid. Fluid is administered to body tissue 140 adjacent conductive tip 111. By way of contrast, in accordance with prior art techniques, fluid is injected through the distal end of an introducer and away from the conductive tip. If the treatment composition is electrically conductive, this delivery of treatment fluid provides first, better conductivity from the conductive tip 111 to tissue surrounding conductive tip 141 and second, greater efficacy of the energy delivered to body tissue 140. Treatment fluid may be delivered to body tissue surrounding conductive tip 111 by either rotating device 100 about the axis of conductive shaft 124 while simultaneously administering treatment fluid through aperture 128 or by rotating device 100 about the axis of conductive shaft 124 to desired orientation to target specific body tissue and subsequently administer treatment fluid through aperture 128. Handle 130 comprises first orientation markings 131 to indicate 180° rotation of device 100 about the axis of shaft 124. Handle 130 also comprises second orientation markings 132 to indicate 90° rotation of device 100 about the axis of shaft 124. The user may use first and/or second orientation markings 131,132 to prevent device 100 from rotating about the axis of shaft 124 while device 100 is inserted through body tissue 140 or to rotate the device about the axis of shaft 124 to a desired orientation. First and second orientation markings 131, 132 may be visual indicators, which may be flush with handle 130, or tactile indicators, which may be textured or raised, so that the user may see or feel the markings as device 100 is inserted into body 140. The proximal end of handle 130 has a strain relief 133 with grip 136 running from the proximal end to the distal end of strain relief 133. Grip 136 is preferably textured such as with parallel ridges to provide points of friction for the user while device 100 is rotated about the axis of shaft 124 and inserted through body 140. Strain relief 133 has a non-round (non-circular) cross-section, which may be square, triangular, or “toothed” like a mechanical gear. Strain relief 133 is tapered with a larger distal outer diameter, to fit with handle 130, and a smaller proximal outer diameter, to fit an extending electrical cable 115 and flexible tubing 113. This taper provides increased grip for the user and reduces slipping of the user's fingers as device 100 is advanced into body 140. Strain relief 133 provides a comfortable handle for the user and may conform to a user's gripping preference. Strain relief 133 is a soft flexible bend relief, which offers support to electrical cable 115 and flexible tubing 113. Electrical cable 115 and flexible tubing 113 extend from handle 130 and strain relief 133 in parallel and close together. Notably, electrical cable 115 and flexible tubing 113 do not extend from handle 130 perpendicular to each other. This arrangement provides comfortable grasp in the user's hand and ease of manipulation of device 100 during placement, rotation, insertion, etc.

Electrical energy may be supplied to conductive tip 111 via conductive shaft 124 from power source control unit 160 via electrical cable 115 and an electrical connector 114. All electrical contacts, except for conductive tip 111, are isolated from the user by a connector pin housing 116. Electrical cable 115 preferably supplies energy to conductive tip 111 via conductive shaft 124. Electrical cable 115 also relays temperature data back to power source control unit 160 to be monitored by a user. In the preferred embodiment of the invention, one conductor in electrical cable 115 is shared between the thermocouple wires and the RF delivery wire. This sharing reduces the overall mass of electrical cable 115 and minimizes the forces and moments applied at handle 130 during placement of device 100 in body tissue 140. It will be understood by a person skilled in the art that separate cables may be used in conjunction with temperature sensor 126. A fluid delivery mechanism 110 can be coupled to fluid delivery interface connection 112 to administer a therapeutic composition. Device 100 therefore may be simultaneously connected to fluid delivery mechanism 110 and power source control unit 160 to treat body 140. Fluid delivery interface connection 112 may be any connector that allows for the flow of fluid from a fluid delivery mechanism, such as a luer type connector, to flexible tubing 113.

In operation, device 100 is inserted into body 140 and placed at target location 141. Proper placement of device 100 is confirmed by stimulating target area 141 by applying electrical energy using conductive tip 111. Anesthetic fluid or another treatment composition can then be administered by actuating fluid delivery mechanism 110. Treatment composition exits fluid delivery mechanism 110 and flows through fluid delivery interface connection 112, flexible tube 113, and the lumen of shaft 124 to conductive tip 111 where it exits through aperture 128. Device 100 obviates the need to use and therefore remove a probe to apply a treatment composition. Fluid delivery mechanism 110 may be pre-connected to fluid delivery interface connection 112 flexibly coupled to shaft 124 without adjusting the position of conductive tip 111. Therefore, after stimulation to confirm proper placement of device 100, manual manipulation of device 100 is minimized and thus the likelihood of shifting of device 100 out of position is decreased. Other methods to confirm placement other than electrical stimulation can also be used, such as taking an impedance measurement or using imaging technology. Flexible tube 113 further decreases the forces acting on handle 130 and shaft 124 when a component on fluid delivery mechanism 110 is actuated to administer the treatment composition, for example, a plunger for a syringe.

After administering the treatment composition, a high frequency electrical current may be applied to target area 141 through conductive tip 111. Return dispersive electrode 150 is provided to create a closed circuit when device 100 is electrically operated in contact with body 140. Notably, since both the fluid delivery mechanism is still connected to the device 100, further delivery of treatment composition simultaneously with the delivery of energy is possible. Temperature sensor 126 is shared with the RF delivery conductor. Preferably, temperature sensor feedback is used to automatically control the RF energy delivered to body tissue 140 to provide safe operation of device without harm to a patient. For example, if the body tissue temperature increases rapidly while applying RF energy as measured by the temperature sensor feedback mechanism, RF energy delivery to body is reduced in real-time to provide a controlled ramp to the desired set temperature. In this manner, the user does not blindly apply RF energy to the body tissue, but is informed, in real-time, of the effects that RF energy delivery has on tissue temperature.

In this embodiment, flexible tube 113 provides the mechanical slack between the fluid delivery interface connection 112 and handle 130 to ensure the fluid delivery system does not introduce added force to the device. Other treatment tools, depending on the procedure, may also be flexibly connected to the device 100. Device 100 may therefore be provided with pre-formed connectors for these treatment tools that are flexibly coupled at or near the proximal end.

In this embodiment, shaft 124 and conductive tip 111 portions are made from a conductive material, for example stainless steel. Insulating coating 122 can be made of any type of insulative material such as Polyethylene Terepthalate (PET) to prevent shaft 124 from delivering the high frequency electrical current to tissue surrounding the shaft 124. This coating can be applied using dip coating, heat shrink coating or any other method that would be understood by a person skilled in the art.

In some embodiments of the invention, to facilitate precise placement of conductive tip 111, conductive tip 111 is distinguishable from the rest of the needle when viewed under X-rays and fluoroscopy by providing a radiopaque marking at the proximal end of the conductive tip 111 or a radiopaque marking along the length of insulating coating 122.

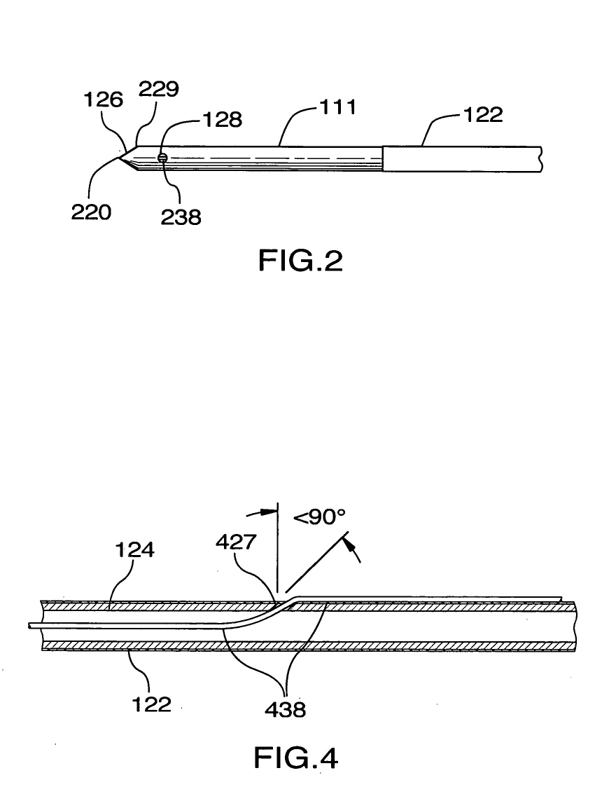

A magnified view of a distal end of the device 100 is shown in FIG. 2. The distal end of the device 100 comprises temperature sensor 126, aperture 128, wiring 238, conductive tip 111 and insulating coating 122. Temperature sensor 126 is welded to the distal end of conductive tip 111 and sharpened to a desired shape, such as a pencil point tip 220, to increase the ease of insertion and reduce the thermal mass of the temperature sensor for faster response. A smaller thermocouple mass, undergoing a change in temperature, requires less time to generate a temperature feedback signal than a larger mass. This decreased delay allows for better automatic control from the power source control unit. As noted, the circumferential edge of aperture 128, on the outer diameter of conductive tip 111, is preferably rounded to prevent cutting of body tissue 140 while device 100 is advanced therethrough. Wiring 238 is electrically coupled to conductive tip 111 and temperature sensor 126. The sharp pencil point tip 220 of temperature sensor 126 provides the user with the ability to penetrate tissue and guide the point 220 through turns and angles with a rounded proximal surface 229 of temperature sensor 126.

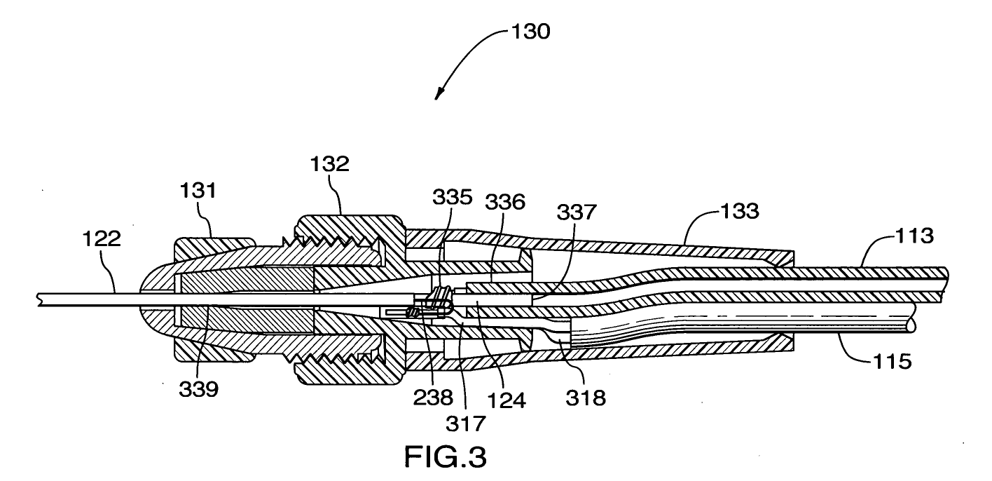

Referring to FIG. 3, a magnified sectional view of handle 130 of device 100 is shown. Handle 130 further comprises a compression gasket 339 to provide radial centering of shaft 124 in handle 130. A solder joint 335 provides electrical coupling between a first conductor 318 of electrical cable 115 and conductive shaft 124. First conductor 318 is shared between the thermocouple wires and the RF delivery wire. Wiring 238 exits the insulating coating 122 and lumen of conductive shaft 124 and is electrically coupled to a second conductor 317 of electrical cable 115. Flexible tube 113 is coupled to conductive shaft 124 and is slid over a proximal end 337 of conductive shaft 124. A junction 336 provides fluid communication from fluid delivery interface connection 112 to aperture 128.

Referring to FIG. 4, a magnified sectional view of proximal end 337 of conductive shaft 124 and insulating coating 122 of device 100 is shown. Thermocouple wiring 438 exits the lumen of conductive shaft 124 through a side aperture 427. Side aperture 427 is preferably angled less than 90° with the axis of conductive shaft 124, as shown in FIG. 4 to minimize bending of wiring 438. This angle provides additional strain relief and protection of the insulation of thermocouple wiring 438 as it exits the lumen, lies parallel to conductive shaft 124 and is covered by insulating coating 122.

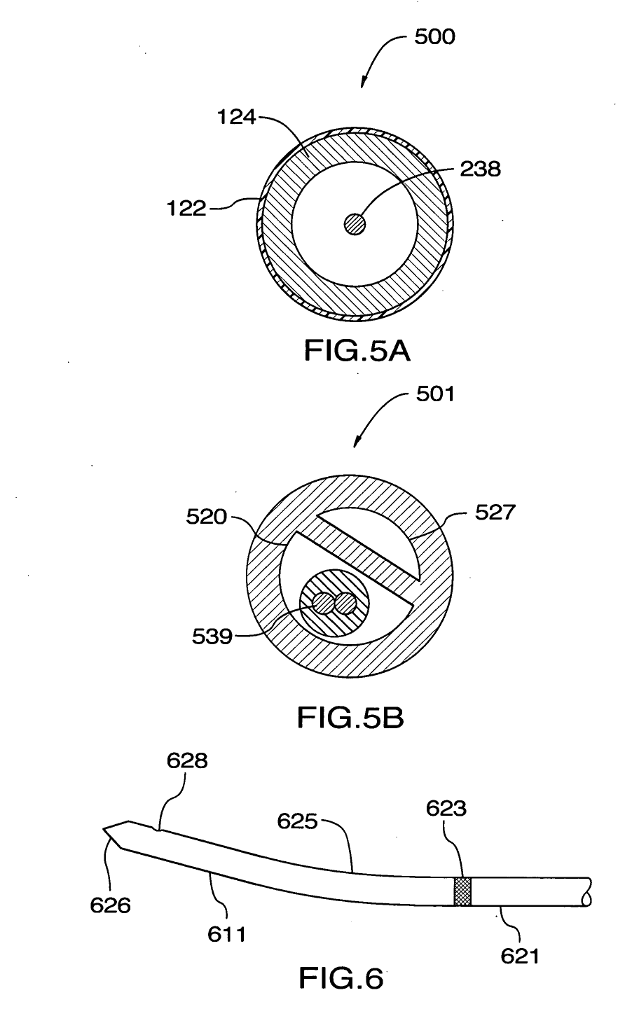

Referring to FIG. 5A, a sectional view of a shaft portion 500 of conductive shaft 124 with insulating coating 122 is shown. Temperature measurement wire or wires 238 run through the lumen defined by the hollow conductive shaft 124. The preferred embodiment of the present invention comprises a single wire 238 electrically coupled to the electrical cable 115, housed in the lumen of conductive shaft 124, and welded to the conductive tip 111 to form temperature sensor 126.

Another embodiment for a shaft 124 of a surgical device aspect of the invention can be seen in FIG. 5B showing a sectional view of a shaft portion 501. This embodiment of shaft portion 501 comprises a first lumen 527 and a second lumen 520. Wiring 539 for temperature sensor 126 and conductive tip 111 of the device run through second lumen 520. First lumen 527 is used as a passage for the injection of a treatment composition. The size of lumen 527 and lumen 520 and the number of lumens required may vary depending on the embodiment.

Another embodiment for a shaft 124 of a surgical device aspect of the invention can be seen in FIG. 6. This embodiment of the shaft 124 comprises a textured surface 621, a radiopaque marker 623, and a curved conductive tip 611. Conductive tip 611 comprises an aperture 628 on the inside of curve 625 and a temperature sensor 626 at the distal end. Textured surface 621 allows for strong adhesion of insulating coating (not shown) to the shaft of the device by increasing the surface area. Radiopaque marker 623 provides visibility of the junction between curved conductive tip 611 and non-conductive portions of the shaft 124 under fluoroscopic imaging. Radiopaque marker 623 defines the distal end point of the insulating coating (not shown) and the proximal start point of conductive tip 623. It should be understood by those skilled in the art that the radiopaque marker 623 may include any arrangement or length of radiopaque marking(s) along the shaft 124 of the device 100. Other arrangements of radiopaque markings may include a series of equidistant markers to indicate insertion depth or may include radiopaque marking along the length of the shaft 124, preferably proximal to the conductive tip. Equidistant depth markings may not necessarily be radiopaque, but may be coloured to contrast against the shaft 124 and be visible to the user. Curved conductive tip 611 provides alternate maneuverability of the shaft 124 while it is advanced through body tissue 140. Having the orientation of aperture 628 on the inside of curve 625 prevents the edge of aperture 628 from cutting body tissue as the shaft 124 is advanced through body tissue 140.

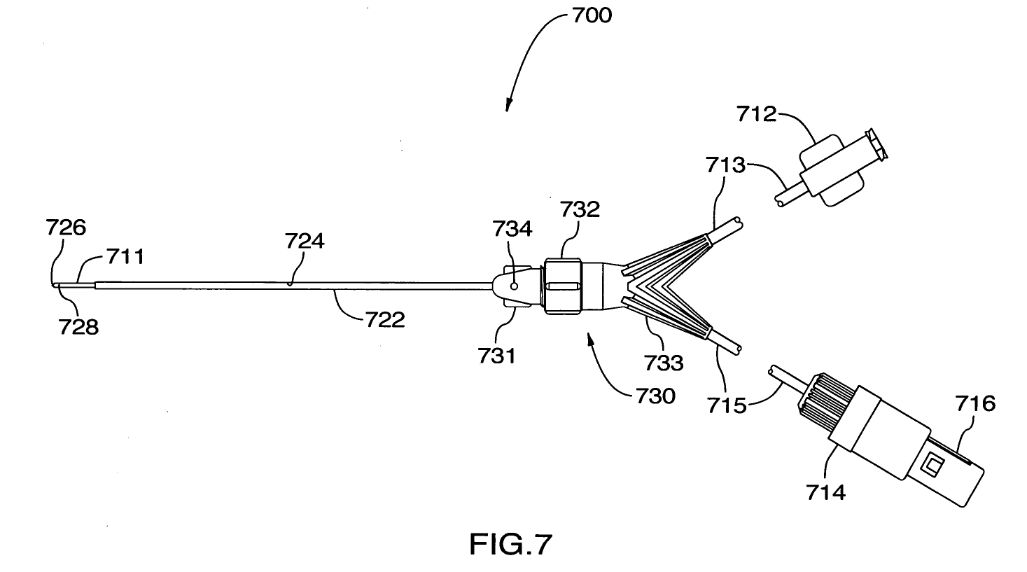

Another embodiment of a surgical device aspect of the invention is shown in FIG. 7. A device 700 has a handle 730 configured to reduce torque. Handle 730 incorporates a “V” shaped housing section 733 having one arm for coupling to an electrical cable 715 and a second arm for coupling to a therapeutic agent tube 713. Electrical cable 715 and therapeutic agent tube 713 extend from the end of handle 730 and are coupled to an electrical connector 714 and a fluid delivery interface connection 712, respectively, to substantially reduce the force transmitted to a conductive tip 711. Similar to device 100, the device 700 in FIG. 7 may also comprise a temperature sensor 726, an insulating coating 722, first orientation markings 731 to indicate 180° rotation, second orientation markings 732 to indicate 90° rotation, and an aperture marker 734 to indicate a location of aperture 728.

It will be understood that the extent to which the tube 713 extends into the handle 730 or onto the lumen of the shaft 724 can vary so long as it is in fluid communication with an aperture 728 at the conductive tip 711.

Though not shown, another embodiment of a surgical device aspect of this invention provides a device, comprising a shaft and a conductive tip constructed from separate components. The shaft could be made of a conductive material and then coated with an insulative material as in the embodiment shown in FIG. 1 or simply made from a non-conductive material such as, but not restricted to, polyetheretherketone (PEEK). The conductive tip is made of a conductive material and connected to the non-conductive shaft. There are various methods in which the conductive tip could be attached to the non-conductive shaft using, but not limiting to, chemical bonding, press fits, screw fits, etc. The wiring for the temperature sensor and the conductive tip may extend through and along a lumen of the shaft and connect to the conductive tip. Alternately, the wiring for the temperature sensor and the conductive tip may be extruded in the walls of the shaft such that the lumen could be used to deliver treatment fluid but would not be required to house the wiring.

This conductive tip can generally therefore serve multiple purposes. The conductive tip can be the site of passage for electric current to the surrounding tissue. It can also be the site for the release of therapeutic agent. Finally, the conductive tip can also house a temperature sensor. In FIG. 2 conductive tip 111 is shown as a pencil point tip with aperture 128 and temperature sensor 126. Other tip geometries, such as a bevel on the end of the conductive tip with a bottom hole and a mid-bevel temperature sensor are also contemplated embodiments (not shown). It should be understood that various other tip shapes, sizes, hole sizes, hole placements, and temperature sensor placements are also considered to be viable options for this invention.

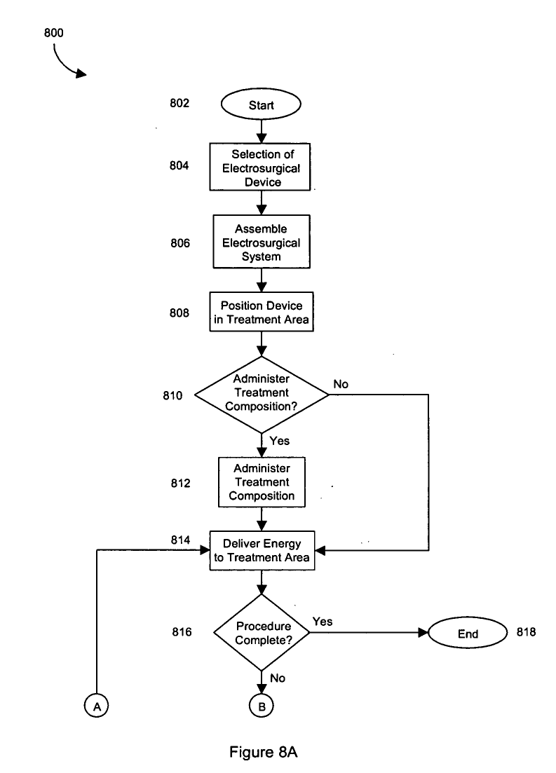

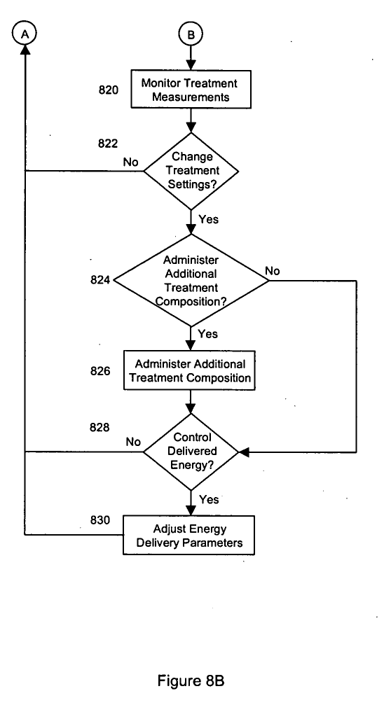

Referring to FIGS. 8A and 8B, a flowchart of operations 800, in accordance with one embodiment of the method aspect of the invention, is illustrated. At step 802, the method for using the electrosurgical device is initiated. At step 804, an electrosurgical device in accordance with the device aspect of the present invention, is selected and obtained for performing the electrosurgical procedure. Reference to electrosurgical device 100 of FIG. 1 in connection with the method aspect of the invention is intended to be exemplary and illustrative only. Selection may thus include choosing one of an assortment of electrosurgical devices for appropriate dimension, such as length, tip shape, active tip length, gauge, etc. Selection may also include obtaining an electrosurgical device from many devices and removing the one electrosurgical device from packaging materials. At step 806, an electrosurgical system, such as system 10 in FIG. 1, is assembled. Assembly may include attachment of fluid delivery mechanism 110 and/or attachment of power source control unit 160, from FIG. 1. Assembly may also include the attachment of return dispersive electrode 150 to body 140. The order in which fluid delivery mechanism 110, power source control unit 160, and return dispersive electrode 150 are assembled, may vary with user preference. Partial assembly of electrosurgical system 10 is also possible; the user may wish to attach return dispersive electrode 150 and power source control unit 160, but leave fluid delivery mechanism 110 detached from the system until a later step. Although the preferred embodiment of the method includes the attachment of power source control unit 160, fluid delivery mechanism 110, and return dispersive electrode 150, it will be understood by those skilled in the art that variations in order of attachment may occur. At step 808, electrosurgical device 100 is positioned in body 140. Positioning may include steps of percutaneous insertion of the distal end of device 100 into body 140. In a preferred embodiment, device 100 is inserted to a final placement position in target area 141 of body 140. However, positioning of device 100 at any placement location may be performed at step 808 and positioning of device 100 in a final location may occur later, but before delivering energy at step 814. At step 810, the user decides whether or not to administer treatment composition fluid to body 140. The decision to administer treatment composition may include factors such as the preference of the physician, allergic reactions to treatment composition, and/or delivery of nerve stimulation energy rather than nerve ablation energy. In the preferred embodiment, the user decides to administer treatment composition fluid and treatment composition fluid is administered via Yes branch to step 812. Administration of treatment composition fluid may include, but is not limited to, injection of sterile water or saline solution to modify electrical properties of body tissue 140, or injection of local anesthetic solution to block, hinder or change the signal propagation of pain from body tissue 140. It will be understood by those skilled in the art that other treatment fluids or combinations of the abovementioned treatment fluids may be injected into body tissue 140 surrounding conductive tip 111 (not shown). In step 810, if the user decides to not administer treatment composition fluid, the user chooses to deliver energy to treatment area via No branch to step 814. Although the preferred embodiment of the method invention is the administration of treatment composition fluid at step 812 prior to delivery of energy to treatment area at step 814, administration of treatment composition fluid may rather occur during and/or after delivery of energy to treatment area.

While energy is being delivered to treatment area at step 814, the user and/or power source control unit 160 monitors for completion of energy delivery to treatment area at step 816. This monitoring may include, but is not limited to, user comparison of elapsed time while energy is being delivered to body tissue compared to the desired or set time for which delivery of energy to treatment area should occur, user choice to terminate energy delivery, logic in power source control unit 160 to end energy delivery upon detection of a system error during energy delivery, and/or measurement in power source control unit 160 to automatically terminate energy delivery upon elapsed time reaching desired time, which is set on power source control unit 160. System error may include, but is not limited to, detection of discontinuity between electrosurgical device 100 and power source control unit 160, high or low impedance between conductive tip 111 and return dispersive electrode 150, delivered power exceeding power limit, etc. If energy delivery to treatment area is complete, the procedure is stopped at step 818 via Yes branch. If energy delivery to treatment area is not complete in step 816, measurements from electrosurgical device 100 at the treatment area are monitored manually by user or automatically by power source control unit 160 at step 820 via No branch from step 816. In the preferred embodiment, temperature measurement of the treatment area is monitored at step 820 monitoring of temperature in treatment area may include, but is not limited to, the measurement of temperature through the use of a temperature sensor, such as a thermocouple or thermistor, feedback to power source control unit 160, and/or temperature data feedback signal to external thermometer, separate from power source control unit 160. In the preferred embodiment of the method invention, temperature of the treatment area is fed back to power source control unit 160 to be used for decision-making at step 822. At step 822, the measured temperature (or other measurement) of the treatment area is compared against predetermined values. These values may include value ranges, threshold values, individual values, etc. The user, who is continuously monitoring the temperature (or other measurement) of the treatment area, may make the comparison of measured temperature (or other measurement) against acceptable/unacceptable values. Alternatively, in the preferred embodiment, power source control unit 160 automatically compares the measured temperature (or other measurement) to predetermined values and makes a decision whether measured temperature is acceptable or unacceptable. Notably at step 820, power source control unit 160 or user may also monitor other measurements related to treatment area such as impedance, power, current, voltage, etc., and use these measurements to make decision(s) at step 822. For example, temperature measurement of treatment area is the preferred measurement for decision-making with the invention. If the measured temperature is acceptable, energy delivery settings are not changed and energy delivery to treatment area continues via No branch back to step 814. If the measured temperature is unacceptable, treatment settings must be changed via Yes branch to step 824. At step 824, the user has the option to administer additional treatment composition fluid via fluid delivery mechanism 110. Administration of additional treatment composition fluid may occur at any time before, during, or after energy is being delivered to treatment area. This administration of additional treatment composition may include abovementioned administration of treatment composition fluid at step 812. Step 824 may also be the initial delivery of treatment composition to treatment area if the user had decided to not administer treatment composition at step 810. If the user decides to administer treatment composition at step 824, additional treatment composition is administered to treatment area via Yes branch to step 826. With the completion of step 826, the user and/or power source control unit 160 has the option to modify electrosurgical system 100 settings to control energy being delivered to treatment area at step 828. If the user decides to not administer treatment composition at step 824, the user and/or power source control unit 160 has (have) the option to modify electrosurgical system 100 settings to control energy being delivered via No branch at step 828. The option to modify electrosurgical system 100 settings, at step 828, may include, but is not limited to, user choice to manually change power setting to increase/decrease temperature of treatment area, user choice to increase/decrease set temperature of system to change lesion size, automatic change in power setting to control temperature of treatment area. At step 828, if the user and/or power source control unit 160 decide(s) not to modify electrosurgical system 100 settings to control energy being delivered, energy continues to be delivered to treatment area via No branch at step 814. If the user decides to manually modify or change the electrosurgical system 100 settings to control energy being delivered to treatment area, system settings are manually adjusted via Yes branch to step 830. Alternatively and in the preferred embodiment, if the power source control unit provides automatic control of energy delivery to treatment area and power source control unit 160 determines that electrosurgical system 100 parameter settings must be adjusted, adjustment occurs via Yes branch to step 830. Automatic control may include the ability of power source control unit 160 to continuously monitor treatment measurements, compare said measurements to acceptable values, ranges, etc., make decisions based on said comparison, modify system parameters to obtain acceptable treatment measurements. Parameter settings may include, but are not limited to, settings for power, current, voltage, temperature, delivery rate(s), time, etc. Said treatment measurements may include, but are not limited to, measurement of impedance, voltage, current, power, temperature, continuity between electrosurgical device 100 and power source control unit, error checking, etc. After all required adjustments are made, manually by user or automatically by power source control unit 160, energy continues to be delivered to treatment area with the new settings at step 814. The energy delivering process from step 814 through to step 830 continues until step 818 is reached, where a system error occurs or elapsed time equals desired set time.

One advantage of the method and apparatus of the present invention is that a decrease in the surgical time is facilitated by allowing for placement of the treatment device, reading of temperature, reading of tissue impedance, therapeutic agent injection and energy activation to be controlled through the same instrument. A separate device, such as an introducer, is not needed to introduce the energy-delivering device to the body. The same device that delivers energy to the body is also used to penetrate the skin and tissue for placement.

The simple design of the apparatus allows it to be manufactured as a one-time use device so as to increase sterility assurance.

The simultaneous delivery of energy and administration of treatment composition to the treatment region of body tissue is facilitated by the surgical device. Since additional equipment is not added for energy delivery, such as a probe, and additional equipment is not removed for composition delivery, such as a stylet, energy and treatment composition may be delivered to the body tissue simultaneously. If the patient is experiencing pain or the conductivity of the tissue surrounding the conductive tip needs to be modified, the user may inject additional treatment composition while energy is delivered to the body tissue via the conductive tip. When a procedure has started and energy is being delivered, the six steps for injecting additional treatment composition of the prior art system can include (1) stopping the energy from being delivered, (2) removing the probe from the introducer, (3) attaching the syringe to the introducer, (4) administering treatment composition, (5) re-inserting the probe in the introducer, and (6) re-starting the delivery of energy. These six steps can be reduced to one step of administering treatment composition while energy is simultaneously delivered, as described at step 826 in FIG. 8B. In accordance with a method aspect of the invention, prior to the start of energy delivery, treatment composition may be administered to the body. Hence, the fluid delivery mechanism, such as a syringe, is attached to the electrosurgical device and is available for use during energy delivery, if the administration of additional treatment composition is required.

Advantageously, the method and apparatus provided may serve to reduce movement of the conductive tip during operation, which may be facilitated for the following reasons. First, the probe and any other treatment tools, such as a syringe for injecting a treatment composition and a temperature sensor, are already or are optionally pre-connected to the device. Therefore only one placement of the device may be required and no items have to be removed or attached during the procedures. The device has a minimally varying mass, as a minimal change in mass may occur during the administering of treatment fluid. The mass of the device does not vary as in the prior art probe and introducer system, where the sequential steps of removing a stylet, inserting a probe, removing a probe, attaching a syringe, administering treatment fluid, removing a syringe, and inserting a probe, produce significant variation in mass and cause changing forces and moments to be applied at the handle and the conductive tip. In addition, a mounting device may be used in conjunction with the electrosurgical device. The mounting device is typically fixed to the surface of the skin and the electrosurgical device is attached to the mounting device. The mounting device serves as a reference platform for the electrosurgical device and allows the user to accurately place the electrosurgical device in body tissue without the concern of changes in placement position. Second, treatment tools are flexibly connected to the device, such as via flexible electrical cable and flexible tubing. The electrical connection interface and fluid delivery connection interface are respectively connected to the electrical cable and flexible tubing at a distance from the device handle so that the forces on the handle or moments on the conductive tip, introduced by the mass of these connections, are minimized. This reduces movement of the conductive tip when manipulation of a treatment tool is necessary during operation, such as actuating a plunger on a syringe or repositioning the power source control unit. Furthermore, a slack in the flexible coupling reduces movement of the device resulting from the weight and balance of the device once it has been inserted.

Although preferred embodiments of the invention have been described herein, it will be understood by those skilled in the art that variations may be made thereto and different embodiments may be combined in any logical manner without departing from the spirit of the invention or the scope of the appended claims.

1. An electrosurgical device for treating animal tissue comprising:
an elongate member having a proximal end, a distal end and a lumen therethrough;
a conductive tip at the distal end for delivering energy to the tissue;
a power connector at the proximal end for flexibly coupling a power source to supply energy to the conductive tip;
a temperature sensor at the distal end; and
at least one fluid delivery interface connection flexibly coupled at the proximal end for coupling a fluid delivery mechanism to deliver a treatment composition through the lumen to the distal end.

2. The electrosurgical device of claim 1, wherein the temperature sensor is located at the conductive tip.

3. The electrosurgical device of claim 1, wherein the temperature sensor comprises a thermocouple.

4. The electrosurgical device of claim 3, wherein the thermocouple is welded to the conductive tip.

5. The electrosurgical device of claim 1, wherein the energy is a high frequency electrical current.

6. The electrosurgical device of claim 1, further comprising at least one pre-formed connector flexibly coupled at or near the proximal end for coupling at least one predetermined treatment tool.

7. The electrosurgical device of claim 1, wherein the conductive tip defines at least one aperture in fluid communication with the lumen and through which the treatment composition exits.

8. The electrosurgical device of claim 7, wherein the at least one aperture is on a side of the conductive tip.

9. The electrosurgical device of claim 8, including a flexible tube connecting the fluid delivery interface connection to the lumen.

10. The electrosurgical device of claim 1, wherein the conductive tip is sharp for piercing through tissue.

11. The electrosurgical device of claim 1, wherein at least a major portion of the elongate member is electrically insulated.

12. The electrosurgical device of claim 11, wherein the elongate member comprises an insulating coating.

13. The electrosurgical device of claim 12 wherein the elongate member comprises a conductive shaft coupled to the conductive tip, said shaft coated with the insulating coating.

14. The electrosurgical device of claim 13, further comprising wiring for delivering energy to the conductive tip, the wiring connected to the conductive shaft at the proximal end.

15. The electrosurgical device of claim 1, wherein the lumen houses wiring to couple the conductive tip to the power source.

16. The electrosurgical device of claim 1, wherein the lumen houses wiring connected to the temperature sensor providing temperature data to a user.

17. The electrosurgical device of claim 1, wherein the elongate member has a second lumen, the second lumen carrying wiring for at least one of the temperature sensor and the conductive tip.

18. The electrosurgical device of claim 1, further comprising a handle at the proximal end coupled to the elongate member.

19. The electrosurgical device of claim 18, wherein the handle couples the power connector and the fluid delivery interface connection to the elongate member.

20. The electrosurgical device of claim 18, wherein the power connector is embedded in the handle.

21. The electrosurgical device of claim 18, wherein the power connector is flexibly coupled to the handle.

22. The electrosurgical device of claim 18, wherein the handle comprises one or more passages in fluid communication with the lumen of the elongate member.

23. The electrosurgical device of claim 22, wherein the one or more passages house at least one of cable, wiring and tubing extending from the handle.

24. The electrosurgical device of claim 1, comprising a radiopaque marker associated with the conductive tip.

25. The electrosurgical device of claim 1, wherein the elongate member is rigid.

26. The electrosurgical device of claim 1, wherein the tissue is neural tissue.

27. The electrosurgical device of claim 1, comprising wiring extruded in a wall of the elongate member, said wiring comprising at least one of the power connector to the conductive tip and the temperature sensor for providing temperature data to a user.

28. The electrosurgical device of claim 1, wherein the device is electrically isolated from a user except for the conductive tip.

29. The electrosurgical device of claim 1, wherein the conductive tip is curved.

30. The electrosurgical device of claim 1, comprising wiring connecting the power connector and the conductive tip to supply energy and connecting the temperature sensor for providing temperature data to a user, said wiring for the conductive tip sharing a wire with the wiring for the temperature sensor.

31. The electrosurgical device of claim 4, wherein the thermocouple is sharpened.

32. The electrosurgical device of claim 8, wherein the at least one aperture has a rounded edge on an outer wall of the conductive tip.

33. The electrosurgical device of claim 8, having a marker associated with the proximal end to indicate the orientation of the at least one aperture on the side of the conductive tip.

34. The electrosurgical device of claim 12, wherein an outer surface of the elongate member is textured for application of the insulating coating.

35. The electrosurgical device of claim 16, wherein the wiring exits the lumen through a side aperture in the lumen at the proximal end.

36. The electrosurgical device of claim 35, wherein the side aperture is defined such that the wiring bends less than 90 degrees relative to the axis of the elongate member.

37. The electrosurgical device of claim 18, wherein the handle has markings to indicate a 180° rotation of the device about a longitudinal axis of the elongate member.

38. The electrosurgical device of claim 18, wherein the handle has markings to indicate a 90° rotation of the device about a longitudinal axis of the elongate member.

39. The electrosurgical device of claim 23, wherein the handle further comprises a strain relief for the at least one of cable, wiring and tubing extending from the handle.

40. The electrosurgical device of claim 39, wherein the strain relief holds the least one of cable, wiring and tubing closely together and in parallel to a longitudinal axis of the handle.

41. The electrosurgical device of claim 39, wherein the strain relief is tapered from the distal end to the proximal end.

42. The electrosurgical device of claim 39, wherein the strain relief is textured.

43. The electrosurgical device of claim 1, wherein the elongate member further comprises a radiopaque marker.

44. The electrosurgical device of claim 43, wherein the radiopaque marker is at a junction between a non-conductive portion of the elongate member and the conductive tip.

45. The electrosurgical device of claim 43, wherein the radiopaque marker is proximal to the conductive tip.

46. The electrosurgical device of claim 1, wherein the elongate member further comprises depth markers.

47. The electrosurgical device of claim 1, whereby the device is insertable and operable without the need to remove any components.

48. A method for delivering energy to a predetermined treatment area of an animal body comprising the steps of:
(i) providing an electrosurgical device comprising:
an elongate member having a proximal end, a distal end and a lumen therethrough;
a conductive tip at the distal end for delivering energy to the tissue;
a power connector at the proximal end for flexibly coupling a power source to supply energy to the conductive tip;
a temperature sensor at the distal end; and
at least one fluid delivery interface connection flexibly coupled at the proximal end for coupling a fluid delivery mechanism to deliver a treatment composition through the lumen to the distal end;
ii) coupling the power source to the power connector;
iii) coupling the fluid delivery mechanism to the fluid delivery interface connection;
iv) positioning the device at or in the vicinity of the treatment area;
v) administering the treatment composition;
vi) delivering energy to the treatment area;
vii) monitoring temperature at the treatment area; and
viii) controlling the delivered energy based on the monitored temperature;
whereby the steps iv)-vi) are performed without the necessity to remove a part of the electrosurgical device from the animal body.

49. The method of claim 48, wherein controlling the delivered energy is automatic.

50. The method of claim 48, further comprising verifying the positioning using at least one of imaging technology, impedance measurement and electrical stimulation.

51. The method of claim 48, wherein the treatment area is a neural structure.

52. The method of claim 48, wherein the energy is high frequency electrical current.

53. The method of claim 48, wherein the positioning includes penetrating at least one of skin and tissue.

54. The method of claim 48, wherein the conductive tip is sharp.

55. The method of claim 48, further comprising, prior to positioning the device, coupling the electrosurgical device to a mounting device that is disposed to be positioned on the animal body.

56. The method of claim 48, wherein the device further comprises depth markings and the device is positioned to a pre-determined depth using the depth markings.

57. The method of claim 48, wherein the device further comprises a side aperture on the conductive tip from which the treatment composition exits and surrounds the conductive tip and adjacent tissue.

58. The method of claim 48, wherein delivering energy to the treatment area and administering the treatment composition occur simultaneously.
